(19) United States
(12) Patent Application Publication
(10) Pub. No.: US 2004/0168232 A1
(43) Pub. Date: Aug. 26, 2004

(54) Rice resistant to sheath blight, bacterial panicle blight, and other diseases

(76) Inventors: Donald E. Groth, Crowley, LA (US); Steven D. Linscombe, Crowley, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/373,927

(22) Filed: Feb. 25, 2003

(51) Int. Cl. A01H 5/00

(52) U.S. Cl. 800/320.2; 800/295; 800/298

(57) ABSTRACT

Disease resistance in rice is disclosed, originally isolated in mutant rice plants designated LM-1. Resistance has been demonstrated against at least the following diseases: sheath blight, bacterial panicle blight, narrow brown leaf spot, and leaf smut. Preliminary results indicate that there may also be resistance to stem rot.

The development of this invention was partially funded by the United States Government under a HATCH grant from the United States Department of Agriculture. The United States Government has certain rights in this invention.

This invention pertains to rice that is resistant to sheath blight, bacterial panicle blight, and other diseases.

Commercial rice plantings are vulnerable to attack by several diseases. The more significant diseases economically include sheath blight, which is caused by the fungus Rhizoctonia solani, and bacterial panicle blight, which is caused by the bacteria Bukholderia glumae. Sheath blight has been reported to be second in economic significance only to rice blast among fungal diseases of rice worldwide. Sheath blight has a major impact on rice yield in the U.S. Gulf of Mexico rice production areas.

The initial symptoms of sheath blight include lesions on sheaths of the lower leaves. Over time, the lesions expand and can spread to upper plant parts. Infected plants can produce poorly filled grain, particularly in the lower portion of the panicle. The fungal pathogen can survive long periods in the soil to infect crops in subsequent years. The wide use of high-yielding, but susceptible varieties has contributed to a rapid increase in sheath blight. In addition, the heavy nitrogen applications that are often used in rice fields can increase the susceptibility of plants to sheath blight. No rice cultivars currently grown in the United States are considered to have adequate field resistance.

The bacteria Burkholderia glumae can produce symptoms that include seedling blight, sheath rot, and panicle blight. As used in the specification and Claims, all these symptoms are considered to be manifestations of a single disease, which will usually be called just “panicle blight.” Panicle blight is a recurring problem in rice-producing areas in the United States. Its incidence has increased in recent years. For example, a recent survey found the disease to be present in about 60 percent of Louisiana rice fields. It was only fairly recently that the cause was identified as a bacterial pathogen, B. glumae. This bacterium is known in Japan as a grain-rofting bacterium that causes seedling rot and grain rot. The critical stage for infection is the heading stage. The disease causes spikelet sterility as well as discoloration of the developing grains and significant yield losses. The disease can develop from seeds infected the previous year, suggesting that it is seedborne. It is also clear that under certain conditions seed infection can inhibit germination and reduce stands. The disease is a particular problem under conditions of unusually hot weather and warm nights.

No commercially useful source of resistance to bacterial panicle blight has previously been available.

While some sources of sheath blight resistance do exist in experimental germplasm, no rice cultivar having agronomically acceptable characteristics is currently available.

Three other diseases in rice, narrow brown leaf spot, leaf smut, and stem rot, probably have a lower economic impact than panicle blight and sheath blight.

Narrow brown leaf spot, caused by the fungus Cercospora janseana or Cercospora oryzae, varies in severity from year to year. It typically becomes most severe as rice approaches maturity, causing premature ripening and yield reduction. As the name suggests, the disease is marked by leaf spots that are brown, relatively long, and relatively narrow. Premature leaf death can occur. Attack on the flag leaf adjacent to the panicle late in the growing season can reduce yield.

Stem rot, caused by the fungus Sclerotium oryzae, typically occurs in patches in fields during later stages of maturity, resulting in premature death and lodging. Infection typically begins near the water line, causing black lesions on the leaf sheath. The lesions grow, and penetrate other parts of the plant. Injury increases as the plants mature, reaching a maximum near harvest. Weakened stalks can break, greatly reducing yield, and interfering with “ratoon” crops, second crops that are sometimes harvested from the same plants later in the season.

Leaf smut, caused by the fungus Entyloma oryzae, is a relatively minor fungal disease in rice, characterized by black spots on leaves. Leaf smut is not generally thought to cause major economic loss to rice growers.

Patent Abstracts of Japan Publication No. 07274752 A2 (1995) discloses a transgenic plant said to exhibit resistance to rice sheath blight or rice bacterial leaf blight, where the transgenic plant is prepared by transformation with a DNA sequence encoding β-1,3-glucanase and a promoter functional in a gramineous plant.

U.S. Pat. No. 5,663,484 discloses basmati-like rice lines that, among other characteristics, were said to be moderately tolerant to sheath blight.

K. McKenzie et al., “Registration of Two Disease-Resistant Germplasm Lines of Rice,” Crop Science, vol. 26, pp. 839-840 (1986) discloses two rice mutants, designated PI 500071 and PI 500072, that were developed from cobalt-60 irradiation of the Vietnamese cultivar Tetep. Tests for resistance to sheath blight were reported, on a 0-9 scale in which 0 denoted healthy plants and 9 denoted severely diseased plants with most tillers dead at maturity. Sheath blight scores were 3 for PI 500071, 5 for PI 500072, 3 for Tetep, and 9 for Lemont. Ratings for narrow brown leaf spot were also reported: 0, 2, 1, and 3, respectively.

E. Nowick et al., “Registration of PR6555 Rice Germplasm Line with Moderate Resistance to Sheath Blight,” Crop Science, vol. 29, pp. 1096-1097 (1989) discloses rice line PR6555, which had moderate resistance to sheath blight, and which was developed by transferring resistance from the Vietnamese cultivar Tetep to the U.S. cultivar Lemont by backcrossing. Tests for resistance to sheath blight were reported, on a 0-9 scale in which 0 denoted healthy plants and 9 denoted death of the whole plant. Sheath blight scores were 4.4±0.3 and 4.2±1.0 for PR6555, 2.2±0.4 and 1.2±0.4 for Tetep, and 6.7±0.9 and 5.9±1.2 for Lemont.

X. Pan et al., “Major Gene, Nonallelic Sheath Blight Resistance from the Rice Cultivars Jasmine 85 and Teqing,” Crop Science, vol. 39, pp. 338-346 (1999) discloses two sources of partial resistance to sheath blight in rice, sources that were shown by crossing tests to be polygenic. Offspring with both resistance genes had a higher level of resistance than either parent. This paper also provides a brief review of the literature concerning other sources of resistance to sheath blight. Tests for resistance to sheath blight were reported, on a 0-9 scale in which 0 denoted healthy plants and 9 denoted death of the whole plant. Average sheath blight scores for different varieties and hybrids ranged from 0.96 for the most resistant hybrids, to 2.53 for the most resistant variety, to 7.73 for the most susceptible variety.

D. Sah et al., “Physiological Races of Cercospora oryzae in the Southern United States,” Plant Disease, vol. 72, pp. 262-264 (1988) discloses tests in which different rice cultivars were found to have differing susceptibility patterns to different races of the fungus that causes narrow brown leaf spot, while the weedy relative of cultivated rice known as “red rice” was resistant to all races of the fungus that were tested.

Wild relatives sometimes appear to exhibit resistance to diseases to which cultivated rice is susceptible, but it is difficult to use these wild relatives as breeding material in developing cultivars.

We have discovered a novel source of disease resistance in rice. This source of novel disease resistance was originally isolated in mutant rice plants. Resistance has been demonstrated against at least the following diseases: sheath blight, bacterial panicle blight, narrow brown leaf spot, and leaf smut. Preliminary results indicate that there may also be resistance to stem rot. By contrast, the “parent” variety from which the mutant was derived, Lemont, is substantially more susceptible to each of these diseases. The novel source of disease resistance may be bred into other lines of rice to produce new varieties, or improved versions of existing varieties, using techniques known in the art of plant breeding, such as crossing and back-crossing.

We have developed a mutant strain of rice (Oryza sativa L.), which we have designated “LM-1.” The mutant was originally obtained by selection conducted on offspring of seed of the variety “Lemont,” seed that had been subjected to cobalt-60 gamma irradiation. Lemont is widely recognized for its high yield, good milling qualities, and excellent agronomic characteristics. However, Lemont is also known for its susceptibility to sheath blight, caused by the fungus Rhizoctonia solani, and its susceptibility to bacterial panicle blight, caused by the bacteria Burkholderia glumae. These two diseases have a major impact on rice farming in the southern United States, and cause significant losses in both yield and quality. Most long-grain rice cultivars are susceptible to sheath blight, and all cultivars are susceptible to bacterial panicle blight to some extent.

Mutations were induced in seed of the variety Lemont by exposure to gamma irradiation. 1.0 kg of seed were subjected to 25 k-rad of gamma irradiation from a Cobalt-60 source at the Nuclear Science Center, Louisiana State University, Baton Rouge, La. prior to planting. The irradiated M₀ seed was planted for seed increase, and the resulting M₁ seed was planted in rows for further seed increase. Selection was conducted on M₂ plants.

Selection was conducted in sheath blight-inoculated rows in the field. Rows were inoculated with R. solani that had been grown on a rice grain: rice hull medium. Twenty-three apparently disease resistant plants were selected from the inoculated M₂ plants for further seed increase and selection. Selection for sheath blight resistance, uniform plant type, and low grain sterility were repeated in generations M₃ through M₇. The level of resistance to sheath blight appeared to be qualitatively similar in each generation, although the incidence of resistance increased at each generation due to Mendelian segregation and selection. Panicle rows were used for advancement, and seed of five panicle rows from the same M₇ family were bulked and designated as “LM-1.”

Subsequently, small plots of seven rows, each 1.2 m long, were planted in the field with LM-1, and the “parent” variety Lemont as a check. Plots were arranged in a randomized complete block design, with five replications. Some plots of each variety were inoculated for sheath blight, and some were inoculated with bacterial panicle blight. Plots were inoculated with 100 mL of R. solani inoculum that had been grown on a moist rice grain: rice hull medium. Inoculation with R. solani occurred at the panicle initiation growth stage.

B. glumae inoculant was grown on nutrient agar in Petri dishes for 3 days; bacterial cells were then suspended in water, adjusted to approximately 10⁷ c.f.u. per mL; and sprayed onto plants at the late boot growth stage using a carbon dioxide-pressurized backpack sprayer.

Plots of LM-1 and of Lemont were replicated four times and arranged in a randomized complete design. Data were collected on overall plant health (expressed as a rating of 0-9, where 0 indicated no disease, and 9 indicated dead plants), extended flag leaf plant height, and days to 50% heading. Plant condition was evaluated by one of the inventors, who did not know at the time of evaluation which plots had received which treatments. (Note that, for various reasons, these numerical scores may not be directly comparable to the disease resistance ratings discussed above in connection with some of the prior references.)

Plant health ratings averaged 4.2 for sheath blight-inoculated LM-1, and 8.0 for sheath blight-inoculated Lemont. The difference was statistically significant at the P=0.05 level.

Plant health ratings averaged 3.0 for panicle blight-inoculated LM-1, and 7.0 for panicle blight-inoculated Lemont. The difference was statistically significant at the P=0.05 level.

In similar tests conducted in replicated, naturally-infected plots, LM-1 was rated 0.8 (very resistant) to narrow brown leaf spot (Cercospora oryzae), compared to 2.5 (resistant) for Lemont, a difference that was statistically significant at the P=0.05 level. LM-1 was rated 2.0 (resistant) to leaf smut (Entyloma oryzae), compared to 4.5 (moderately susceptible) for Lemont, a difference that was statistically significant at the P=0.05 level.

Preliminary results indicate that LM-1 may also exhibit resistance to stem rot, to which Lemont is susceptible. Additional testing will be conducted on stem rot resistance.

In plots that had not been inoculated with pathogens, the average height of LM-1 plants was 77 cm, compared with 76 cm for Lemont. LM-1 headed in an average of 75 days, compared with 72 days for Lemont. In general, the plant type of LM-1 was-very similar to that of Lemont. Occasional tall off-types were produced in the LM-1 plots, but these plants appeared to have similar levels of resistance to sheath blight and panicle blight as the other LM-1 plants.

The resistance to sheath blight was expressed uniquely. The LM-1 plants remained green and healthy several weeks after physiological maturity. By contrast, in germplasm that has previously been reported to show resistance to sheath blight, for example in the Tetep, PR6555, PI 500071, and PI 50072 lines discussed above, the plants have not remained healthy past physiological maturity.

Genetic tests will be conducted to ascertain how many genes are involved in the observed disease resistance. Although such tests had not been conducted as of the filing date of the present application, there is circumstantial evidence suggesting that a single mutation event was responsible both for resistance to sheath blight and for resistance to panicle blight. Not only would it be statistically improbable that two independent, beneficial mutations would occur simultaneously, but selection for disease resistance in the M₂ through M₇ generations was based on resistance to sheath blight only. There was no selection for panicle blight resistance, and yet the same plants that displayed resistance to sheath blight also showed resistance to panicle blight. Without wishing to be bound by this theory, these observations suggest that a single mutation event was likely responsible both for resistance to sheath blight and for resistance to panicle blight. If this hypothesis is correct, then the results reported here are particularly surprising. To the inventors' knowledge, there has been no previously-reported, single source of resistance to both sheath blight and panicle blight—whether in a cultivar, an experimental breeding line, or a wild relative of rice.

A sample of the seed from rice line LM-1 was deposited under the Budapest Treaty with the American Type Culture Collection (ATCC), address 10801 University Boulevard, Manassas, Va. 20110-2209 on 19 Feb. 2003, and was assigned ATCC Accession No. PTA-nnnn. This deposit was made pursuant to a contract between ATCC and the assignee of this patent application, Board of Supervisors of Louisiana State University and Agricultural and Mechanical College. The contract with ATCC provides for the permanent and unrestricted availability of these seeds or the progeny of these seeds to the public on the issuance of the U.S. patent describing and identifying the deposit or the publication or the laying open to the public of any U.S. or foreign patent application, whichever comes first, and for the availability of these seeds to one determined by the U.S. Commissioner of Patents and Trademarks (or by any counterpart to the Commissioner in any patent office in any other country) to be entitled thereto under pertinent statutes and regulations. The assignee of the present application has agreed that if any of the seeds on deposit should become nonviable or be lost ordestroyed when cultivated under suitable conditions, they will be promptly replaced on notification with a viable sample of the same seeds.

### MISCELLANEOUS

The agronomic traits of the novel rice line should make the LM-1 line directly useful in commercial rice fields. In addition, through routine breeding practices known in the art, LM-1 and its progeny will be used as breeding material to produce other disease-resistant varieties and hybrids for commercial use. Crossing and back-crossing resistant plants with other germplasm through breeding techniques known in the art will yield additional disease-resistant varieties and hybrids having good productivity and other agronomically desirable properties.

The novel source of disease resistance was not prepared by transgenic means. It may be introduced into other rice germplasm through traditional, non-transgenic breeding techniques.

If desired, the source of disease resistance might also be introduced into other rice germplasm through transgenic means, to accelerate what could also be accomplished through more traditional breeding techniques. Even if the latter approach were taken, the resulting plants would probably not be considered “genetically modified organisms,” in that they need not incorporate genetic material from another species. Even marker genes need not be employed, as selection for disease resistance may be used directly to identify successful transformation events. Alternatively, other DNA sequences might be transformed into the novel germplasm, such as sequences encoding herbicide resistance.

Thus rice plants prepared in accordance with the present invention in general are not “genetically modified organisms,” although it would also be possible to prepare genetically modified rice that incorporates the present invention. “Genetically modified organisms” are controversial among certain segments of society, so it can be advantageous to practice the present invention without employing genetically modified organisms.

The following definitions should be understood to apply throughout the specification and claims, unless otherwise clearly indicated by context.

The term “plant” is intended to encompass plants at any stage of maturity, as well as any cells, tissues, or organs taken or derived from any such plant, including without limitation any embryos, seeds, leaves, stems, flowers, roots, single cells, gametes, anther cultures, callus cultures, suspension cultures, other tissue cultures, or protoplasts.

Unless otherwise clearly indicated by context, the “progeny” of a plant includes a plant of any subsequent generation whose ancestry can be traced to that plant.

Unless otherwise clearly indicated by context, a “derivative” of a disease-resistant plant includes both the progeny of that disease-resistant plant, as the term “progeny” is defined above; and also any mutant, recombinant, or genetically-engineered derivative of that plant, in which one or more of the disease-resistance characteristics of the original resistant plant have been transferred to the derivative plant. Thus a “derivative” of a disease-resistant rice plant would include, by way of example and not limitation, any of the following plants that express resistance to one or more of the same diseases: F₁ progeny plants, F₂ progeny plants, F₃₀ progeny plants, and a transgenic rice plant transformed with a disease resistance gene from the resistant rice plant.

The term “resistant” or “resistance,” as used herein, is also intended to encompass “tolerant” plants, i.e., those plants that phenotypically evidence adverse, but not lethal, reactions to one or more diseases—particularly where ultimate crop yields are unaffected, or are affected substantially less than crop yields are affected in susceptible rice lines in response to the same pathogen. “Resistant” or “resistance” should accordingly be understood to be a relative term, to be interpreted by comparison to the susceptibility to a particular disease of United States rice cultivars in commerce as of the filing date of this patent application.

The complete disclosures of all references cited in this specification are hereby incorporated by reference. In the event of an otherwise irreconcilable conflict, however, the present specification shall control.

What is claimed:

1. A rice plant wherein:
(a) said plant is resistant to one or more of the following diseases: sheath blight, bacterial panicle blight, stem rot, narrow brown leaf spot, and leaf smut; and
(b) said plant is a derivative of the plant with ATCC accession number PTA-nnnn; and
(c) said plant has the disease resistance characteristics of the plant with ATCC accession number PTA-nnnn against one or more of the following diseases: sheath blight, bacterial panicle blight, stem rot, narrow brown leaf spot, and leaf smut.

2. A rice plant as recited in claim 1, wherein said plant is the plant with ATCC accession number PTA-nnnn, or is any progeny of the plant with ATCC accession number PTA-nnnn; wherein said plant has the disease resistance characteristics of the plant with ATCC accession number PTA-nnnn to sheath blight, to bacterial panicle blight, or to both.

3. A rice plant as recited in claim 1, wherein said plant is resistant to sheath blight.

4. A rice plant as recited in claim 1, wherein said plant is resistant to bacterial panicle blight.

5. A rice plant as recited in claim 1, wherein said plant is resistant to stem rot.

6. A rice plant as recited in claim 1, wherein said plant is resistant to narrow brown leaf spot.

7. A rice plant as recited in claim 1, wherein said plant is resistant to leaf smut.

8. A rice plant as recited in claim 1, wherein said plant is resistant to sheath blight and to bacterial panicle blight.

9. A rice plant as recited in claim 1, wherein said plant is resistant to sheath blight, to bacterial panicle blight, to narrow brown leaf spot, and to leaf smut.

10. A rice plant as recited in claim 1, wherein said plant is the plant with ATCC accession number PTA-nnnn, or is any progeny of the plant with ATCC accession number PTA-nnnn; wherein said plant has the disease resistance characteristics of the plant with ATCC accession number PTA-nnnn to sheath blight, and to bacterial panicle blight.
